(19) United States
(12) Patent Application Publication
(10) Pub. No.: US 2023/0022232 A1
(43) Pub. Date: Jan. 26, 2023

(54) Device for the Enucleation of Intracorporeal Tissue Regions

(71) Applicants: Arkadiusz Miernik, Freiburg (DE); Medtecton GmbH, Luzem (CH); ALBERT-LUD-WINGS-UNIVERSITÄ FREIBURG, Freiburg (DE)

(72) Inventors: Arkadiusz MIERNIK, Freiburg (DE); Kurt RUFFIEUX, Luzern (CH)

(21) Appl. No.: 17/787,963

(22) PCT Filed: Dec. 22, 2020
(86) PCT No.: PCT/EP2020/087639

§ 371 Date: Jun. 22, 2022

(30) Foreign Application Priority Data
Dec. 23, 2019 (DE) 10 2019 220 537.2

(51) Int. Cl. A61B 18/14

(52) U.S. Cl. CPC A61B 18/1485; A61B 2018/00547

(57) ABSTRACT

The invention is a device for the enucleation of intracorporeal tissue regions, in particular of the prostate, with a probe at whose distal end at least one freely accessible electrode body is mounted to which electrical energy can be applied via at least one electrical line running in the longitudinal extent of the probe. The electrode body has a dome-shaped electrode surface and has cross-sectional surfaces which are orientated orthogonally to the longitudinal extent of the probe surface areas along a first axial portion which contains a distal dome end of the electrode body, which increase continuously as the distance from the distal dome end increases.

### CROSS REFERENCE TO RELATED APPLICATIONS

Reference is made to PCT/EP2020/087639 filed Dec. 22, 2020, designating the United States, and German Application No. 10 2019 220 537.2 filed Dec. 23, 2019, which are incorporated herein by reference in their entirety.

### BACKGROUND OF THE INVENTION

### Field of the Invention

The invention relates to a device for the enucleation of intracorporeal tissue regions, in particular the prostate, with a probe at whose distal end at least one freely accessible electrode body line is mounted, to which electrical energy can be applied via at least one electrical line running in the longitudinal extent of the probe.

### Description of the Prior Art

Devices for the enucleation of intracorporeal tissue regions are mainly used for the purpose of treating benign enlargements of the prostate, known as benign prostate hyperplasia (BHP). Widely used for this are endoscopic instruments which in a minimally invasive manner are positioned transurethrally, i.e. through the urethra, intracorporeally at the location of the prostate to be treated in order to completely or incompletely remove benignly enlarged areas of the prostate. Used for performing the minimally invasive surgical procedure, also known as transurethral resection of the prostate, TURP in short, are so-called resectoscopes (see, for example, document DE 935391 B.) which for the purpose of local tissue removal comprise at least one working channel, through which can be fed a probe-like electrode catheter that has a distal electrode tip designed in the form of a loop, to which HF alternating current can be applied, resulting in heating of the electrode loop with which local thermally-induced tissue layer separation is possible. In order to ensure good optical monitoring during the tissue ablation, during the operation irrigation fluid is continuously applied and removed by suction via the resectoscope. The irrigation fluid is electrolyte-free in the case of so-called monopolar resections, whereas when using bipolar resectoscopes, isotonic saline solutions are deployed. HF mono- or bipolar resection of the prostate is seen as the gold standard in the treatment of BPH, even though it also has some disadvantages. Thus, with this treatment method, the resection of the glandular tissue benignly forming on the prostate is not usually complete and the procedure times required for tissue removal are long, particularly in the case of larger adenomas, and result in a considerable burden for the patient. Moreover, in some cases, the required irrigation of the tissue region being treated in the context of a monopolar resectoscopy with hypotonic irrigation solution leads to so-called TUR syndrome that results in ingressions into the vascular system and can cause possible complications such as pulmonary oedema, cerebral oedema, haemolysis and kidney failure.

Newer transurethral endoscopic enucleation techniques using laser-based resectoscopes, in particular using so-called holmium laser enucleation of the prostate, HoLEP in short, show better clinical results. In particular, as well as the treatment of very large adenomas, laser-based prostate enucleation allows layer-specific preparation, through which sparing layer separation between the prostate capsule and the enlarged internal gland or the adenoma is made possible. This procedure allows for particularly low-bleeding surgery in comparison with conventional methods based on the use of the aforementioned HF electrode loops. In contrast to the known conventional techniques, laser-based enucleation offers complete, fine-tissue separation and preparation of the tissue to be removed.

Due to the high costs, and the large volume of the devices, which have to be positioned accordingly in the operating room, it is only possible to provide and accommodate such systems at a small number of locations.

Document U.S. Pat. No. 10,470,837 describes a device and a method of treating rhinitis, that is chronic inflammation of the nasal mucous membrane, with which it is possible to ablate the nerve branch from the nasal nerves located in the posterior nasal cavity. A flexibly designed surgical probe enables it to be introduced and positioned in the submucosal space of a lateral nasal wall, where neuroablation of the posterior nasal nerve can be performed with the surgical probe. At its distal probe tip, the probe device comprises a lamp that shines through the submucosal tissue, making it possible for the surgeon to visualize the submucosal position of the distal end of the surgical probe from the interior of the nasal cavity. In one form of embodiment the probe tip is rounded and comprises an HF electrode assembly for the local generation of Joule heating.

Document DE 696 36 885 T2 discloses a surgical system with a cooled electrode tip. Integrated along the hollow electrode body, the distal end, which can be pointed or rounded, is a cooling system through which the amount of heat that can be applied by the electrode assembly on the distal area to the intracorporeal environment can be controlled, in order to avoid overheating effects.

Document DE 10 2015 119 694 A1 describes an electrosurgical system for the resection of body tissue that comprises at least two electrodes, to which high-voltage pulses can be applied by means of a high-voltage supply unit, for the purpose of brief gas bubble formation within a fluid in contact with the electrosurgical system.

The electrode stimulation probe set out in document DE 88 07 820 U1 is shaped and sized to match the anatomical features of the human sphincter region for the treatment of bladder or anal incontinence as well as hemorrhoids.

Document WO 02/098312 A2 discloses a probe arrangement with a distal probe tip, in the form of a puncture needle tip, for the electrothermal coagulation of tissue, which has two electrodes, one of which is connected to an internal conductor and the other to an external conductor. Internal and external conductors of the probe arrangement are electrically insulated from each other. The inner conductor is also selected so that the bending stiffness of the probe arrangement is increased.

DE 10 2017 100 409 A1 discloses an electrosurgical device which comprises a handle on which is mounted a shaft that is rotatable in a controlled manner.

### SUMMARY OF THE INVENTION

The invention is a device for the enucleation of intracorporeal tissue regions, in particular of the prostate, with a probe at whose distal end at least one freely accessible electrode body is mounted to which electrical energy can be applied via at least one electrical line running in the longitudinal extent of the probe, in such a way that the disadvantages that were previously linked to HF mono- or bipolar resection of the prostate are avoided and comparable surgical properties and results to those achievable with laser-based endoscopic enucleation of the prostate, EEP in short, should be made possible. More particularly, a cost-effective and qualitatively equivalent alternative to complex laser-based EEP is to be created.

In accordance with the invention, the device for the enucleation of intracorporeal tissue regions, particularly for treating benign prostate hyperplasia, has a dome-shaped electrode surface and has cross-sectional surfaces which are orientated orthogonally to the longitudinal extent of the probe and whose surface areas along a first axial portion which contains the distal dome end of the electrode body, increase continuously as the distance from the distal dome end increases. In addition, the cross-sectional surfaces are each limited by a peripheral edge, each point of which is always differentiable, that is smooth and has no edges or corners. Furthermore, at least in the area of the electrode body distally connected to the probe, the probe has a bending stiffness that under the effect of a bending moment of at least 0.1 Nm acting on the electrode body transversely to the longitudinal extent of the probe, guarantees dimensional stability of the probe.

The device according to the invention is based on largely completely replicating the handling and therapeutic effect of a laser catheter designed for the purpose of prostate enucleation, through a probe that is capable of electrical ablation and coagulation of tissue layers or regions and can also be used in connection with a resectoscope.

For this, the dome-shaped electrode body mounted on the distal end of the probe with the electrode body surface having sliding properties, that are completely smooth and without undercuts or edges, so that through distal advancing or lateral movement of the probe by way of the dome-shaped, smoothly configured electrode body, as atraumatic and blunt preparation of the tissue regions to be treated as possible is enabled. Through small and distal or lateral advancement, the dome-shaped electrode body is able to undo or separate tissue layers through a purely mechanical displacement process without, or without appreciable, lesion formation.

The probe configuration according to the invention is, among other things, based on the knowledge that the sparing, laser-based tissue separation process, that is HoLEP, is essentially dependent on the beam quality and impulse of the laser beam emitted from the laser catheter end. In addition to the beam power, the beam diameter, the beam divergence as well as wavelength and frequency, the beam intensity distribution and the beam interaction with the energy-absorbing medium along the beam cross-section play a role in the interaction between the laser beam, possibly the resulting formation of gas bubbles, and the tissue regions to be penetrated or prepared for the purpose of precise, time-efficient and sparing separation of the adenoma from the prostate.

High-quality laser beams exhibit a minimal beam divergence and have a beam intensity distribution along the beam cross-section in the form of a Gaussian curve, for which reason such laser beams are also known as Gaussian beams. According to current knowledge, this spatial beam intensity distribution and the specific pattern of the spatial energy distribution at the site of the energy application appear to be the preconditions for the advantages associated with laser-based prostate enucleation, which forms the inspiration and basis for the spatial design of the electrode surface, in accordance with the invention, of the electrode body mounted on the distal end of the probe.

The dome-shaped design of the electrode body is, among other things, inspired and motivated by the spatial shape of the radiation intensity profile of a Gaussian beam, and through sparing distal or lateral movement of the probe, allows the blunt preparation of tissue regions or layers. As the electrode body can also be supplied with electrical energy via at least one electrical line, the tissue can, if necessary, also be at least one of severed, ablated and coagulated with electrical support.

The electrode body, which is preferably made of a metal or metal alloy, is either a monopolar electrode connected with one electrical line running along the probe, or a bipolar electrode connected with two electrical lines running along the probe. The innovative probe can be used in conventional resectoscopes and can be connected to HF devices normally present in the operating room. The device according to the invention allows precise, time-efficient and sparing treatment of the prostate through the urethra and, as in the case of laser-based resectoscopes, increases the operative safety and is able to significantly reduce the complication rates in comparison with conventional electrical prostate resection methods (TURP). The device according to the invention is also cost-effective and has the potential for positive and health economic effects on the entire health care system.

Preferably, the dome-shaped electrode surface of the electrode body at least along the first axial portion is replicated as a spatial radiation intensity distribution of a laser beam with a Gaussian intensity distribution, a paraboloid or ellipsoid. Also conceivable and implementable are spatial shapes diverging therefrom, in which the cross-sectional surfaces of the electrode body along each first axial portion are each surrounded by a peripheral edge, which is either exclusively curved or has curved and straight peripheral edge sections, wherein the transitions from curved to straight peripheral edge sections are smooth, that is continuously differentiable. The described curved design allows the operator to work in a preferred direction through rotating the instrument.

Conversely, looking at the longitudinal sections through the electrode body orientated orthogonally to the individual cross-sectional surfaces, a preferred embodiment of the electrode body exclusively has longitudinal sections, which are each delimited by a peripheral contour, also continuously differentiable at each location, that is are smooth in design. Preferably, the peripheral contours delimiting the individual longitudinal sections are described by a pitch circle, a parabola, a partial ellipse or a partial oval. An essential characteristic of the electrode surface designed in a dome-shape in accordance with the invention, is its smooth, outwardly appearing shape, which is suitable for separating tissue regions, in particular tissue layers, in a sparing, mechanical displacement process determined by the shape of the electrode body, in a manner that is lesion-free or largely lesion-free. Sharp-edged surface contours, or contours with small radii, as are usual in conventional electrode loops, are expressly ruled out for the shaping of the electrode body, in order to reach or mechanically advance between two anatomical tissue layers by way of the electrode body in as lesion-free a manner as possible.

In a further preferred form of embodiment, in the area of its distal end, the dome-shaped electrode body has a mamilla-like, also smooth and dome-shaped projection through which the mechanical separation process is sparingly supported when distally advancing the probe designed in accordance with the invention. Further explanations in relation to this can gleaned by way of the following illustrations.

Proximally adjoining the smooth, dome-shaped electrode body, is a second axial portion, along which the cross-sectional surfaces orientated orthogonally to the longitudinal extent of the electrode body remain the same as the distance from the distal dome end increases. Preferably the cross-sectional surfaces along the second axial portion are circular so that in this area the electrode body takes on a straight cylindrical outer shape.

The electrode in accordance with the invention is rigidly connected to the probe which is preferably configured as a hollow cannula and is capable of transmitting axial thrust or pressure forces as well as bending forces. The rigid joining of the electrode body on the hollow cannula preferably takes place by way of a biocompatible, electrically insulating joint in the form of a mechanical connecting piece, made, for example, of ceramic or glass or an epoxide resin-based cast component, which has solidified for connection purposes.

The probe, configured as a hollow cannula, also possesses high bending strength in order to withstand mechanical loads acting on the electrode body transversely to the longitudinal extent of the probe without any changes in shape. Such transverse leads can occur, for example, if the operator moves the electrode body laterally or perpendicularly to the longitudinal extent of the probe in order to remove or separate tissue. In this case, no or only minimal deformations, must occur, preferably along the entire probe, but at least in the distal region of the probe, that extends out of the resectoscope on the distal side for the purpose of blunt preparation. In order to ensure dimensional stability, it is evident to produce the probe, in the form of a hollow cannula, of a metallic material, for example instrument steel, or a robust, rigid, preferably fiber-reinforced plastic with a suitably thickly selected hollow cannula wall thickness.

Thus, at least in the distal region, the probe has a stiffness or bending stiffness that is such that under the effect of a bending moment on the probe resulting from a force acting on the electrode body transversely to the longitudinal extent of the probe while the probe is mechanically firmly clamped in at distance of at least 30 mm from the distal probe tip—which corresponds to typical probe guiding along and through a resectoscope—the distal probe tip is deflected a maximum of 2 mm transversely to the longitudinal extent of the probe, and in no case does plastic deformation of the probe occur. With a deflection of 2 mm, the applied force is measured, and the bending moment (F×1) is calculated. The probe is designed in such a way, that with a bending moment of 0.1 Nm, preferably of at least 0.2 Nm, particularly preferably of at least 0.3 Nm, the above requirements for the dimensional stability of the probe remain guaranteed.

Preferably all surfaces, that is both the surface of the electrode body and that of the joint, as well as of the proximally adjoining rigid hollow cannula are designed to be low-friction, providing a smooth surface and surface sliding. It is useful for at least the electrode surface of the electrode body to be polished or honed.

The device according to the invention can, in parts or completely, be provided with a surface finish, preferably in the form of a coating, that increases sliding ability. The coating may be, for example, of PTFE, polyurethane, polysiloxane or a hydrogel, or polymer with slide resistance-reducing additives, or a combination thereof.

Through the mechanically stiff or rigid connection between the hollow cannula, joint and electrode body, mechanical force transmission is possible along the probe in the axial and lateral direction onto the electrode body, with which sparing and targeted tissue separation in the form of atraumatic, blunt preparation becomes possible. The term “blunt preparation” is taken to mean forcing apart two anatomical structures that are connected to each other by at least one of a connective tissue and a vascularised or avascularised layer. In this way an artificial space is created which can be formed under normal biophysiological and anatomical conditions. The blunt preparation process is exclusively initiated by mechanical movement in the distally forward direction as well as at least one of lateral swinging to and fro and rotary movements of the electrode body about the longitudinal axis of the probe or hollow cannula by an operator in the intracorporeal target tissue region. If required, the electrically conductive electrode body can, through being supplied with high-frequency current, develop at least one of an additional cutting, ablation and/or coagulation effect within the tissue region.

In order for the operator to be able to carry out the process of pushing apart two anatomical layer structures in an as tissue-sparing a manner as possible, effectively but finely dosed and directionally selectively, spatial shapes for embodying the electrode body along the first electrode section are particularly suitable that deviate from axial symmetry about the longitudinal axis of the probe and have two-dimensional cross-sectional shapes that are formed flattened along one of two cross-sectional axes orientated orthogonally to each other, like the shape of a spatula. Through the flattening of the otherwise round, that is edge-free electrode body shape, flat pushing apart of two anatomical layer structures is better possible, in that the respectively flattened electrode body surfaces which are opposite each other along a cross-sectional axis, are guided in parallel between the layer structures to be separated when advancing the probe.

Preferably, but not necessarily, the flattened electrode body surfaces are each symmetrical to a cross-sectional axis, that is both electrode body surfaces have at least one flat surface area or both electrode bodies have at least one convexly curved surface area. It is also conceivable that one electrode body surface is convexly curved and the other electrode body surface is concavely curved, or that one of the two electrode body surfaces has at least one curved surface area and the other electrode body surface has at least one flat surface area.

In the case of asymmetrical flattening of the electrode body, one of the two electrode body surfaces preferably has a curved surface shape in the longitudinal extent to the longitudinal axis of the probe, along which the at least one center of curvature is contained. In contrast, the opposite electrode body surface is preferably largely straight. Of course, flattened electrode body surface shapes deviating therefore are conceivable, which allow effective advancing between and pushing apart of two tissue layers.

Through asymmetrical flattening of the electrode body, through alternating bidirectional rotation of the electrode body about the longitudinal axis of the probe, tissue-displacing tipping movements acting on the tissue surroundings become possible for the operator when advancing the probe, which can in some cases support the process of pushing apart two anatomical layer structures.

In addition, asymmetrical flattening of the electrode body, allows the possibility of asymmetrically designing electrode contact surfaces that come into contact with the tissue.

For example, the largely straight, flattened electrode body shape set out in the previous example of embodiment can be designed to be electrically conductive, whereas the opposite, curved electrode body shape is at least not completely electrically conductive and, for example, is locally covered with an electrically insulating layer, or vice versa.

The device according to the invention is particularly suitable for surgical use in connection with a resectoscope which is known per se, and in addition to a working channel for feeding through the probe according to the invention, envisages further at least one of working and irrigation channels, through which the surgeon can optically monitor the performance of a transurethral prostate enucleation. Preferably, for the purpose of a centered and position-defined guiding of the probe along the working channel of the resectoscope, at least one guide sleeve is applied along the hollow cannula, which both acts as a centering and sliding element and also as a feedthrough element for at least one further medical instrument in parallel to the probe through the working channel.

### BRIEF DESCRIPTION OF THE DRAWINGS

As an example, the invention will be described below, without restricting the general inventive concept, by way of examples of embodiment with reference to the drawings. In these:

FIGS. 1a, b show a side view and top view from above of a device in accordance with the invention;

FIGS. 2a, b show an example of embodiment of an electrode body in longitudinal section and viewed from below;

FIGS. 3a, b show an example of embodiment of an electrode body with a distal, additional dome structure;

FIGS. 4-7 show alternative spatial shapes of the electrode body along the first axial section;

FIGS. 8a-e show a longitudinal section a), longitudinal view rotated about 90° b) of an electrode body mounted on the probe, as well as cross-sections c), d), e);

FIGS. 9a-e show longitudinal section a), longitudinal view rotated about 90° b) of an alternative form of embodiment of an electrode body distally mounted on the probe as well as cross-sections c), d), e);

FIGS. 10a-c show a longitudinal section of an electrode body a), distally mounted on the probe, cross-sections b), c);

FIGS. 11a, b show a longitudinal section of an electrode body a) distally mounted on the probe, cross-section b); and

FIG. 12 shows a longitudinal section of an electrode body distally mounted on the probe.

### DETAILED DESCRIPTION OF THE INVENTION

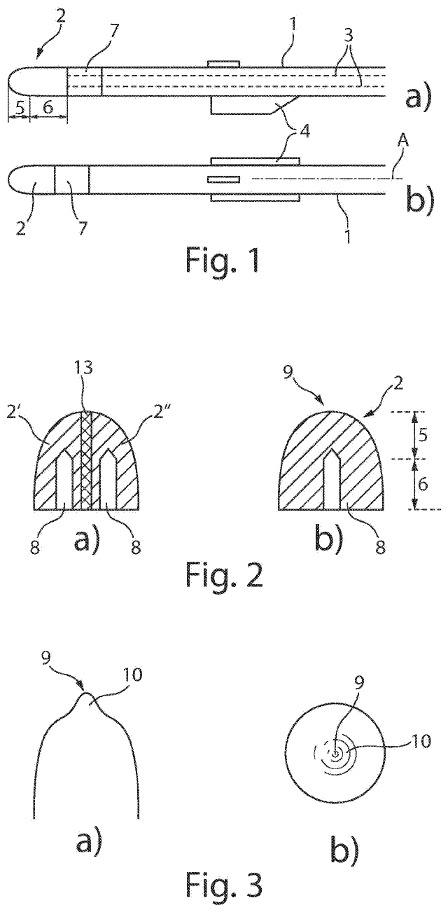

FIGS. 1a, b show a side view and view to a device for the enucleation of intracorporeal tissue regions. The device comprises a probe 1 configured as a hollow cannula, at whose distal end a freely accessible electrode body 2 is mounted. The electrode body 2 is connected to at least one, preferably two electrical lines 3, which extend proximally within the probe 1 and are configured to be connectable to an electrical energy source, which is not shown. Typically, a guide sleeve 4 is attached along the probe 1 which acts as a centering and sliding element within and along a working channel of a resectoscope, which is not shown in more detail, and also allows the feeding through of a medical instrument, which is an optical conductor. The electrically conductive electrode body 2 made of metal or a metallic material, comprises at least one axial portion 6, which has a dome-shaped electrode surface 5, which is spherical in the illustrated example of embodiment. In accordance with FIG. 1, the electrode body 2 comprises a second axial portion 6, which is configured in straight cylindrical form and seamlessly and smoothly adjoins the first axial portion 5.

The metallic electrode body 2 is firmly connected to probe 1, designed as a hollow cylinder, by way of a biocompatible, electrically insulating joint 7. The joint 7 is, for example, designed as a molded body for connection between the electrode body and the hollow cannula.

The curvature of the electrode surface of the electrode body 2 in the first axial portion 5 is constructively determined and optimally selected for the process of blunt preparation. In addition, preferred electrode surface geometries for creating the electrode body 2 are described.

In FIG. 2a, a longitudinal section through a bipolar electrode body 2 is shown, which for the purpose of electrical contacting envisages two electrical contact sleeves 8 into which the ends of the electrical lines 3 enter and are firmly connected to the electrode body 2. Additionally, the electrode body 2 is separated by an electrically insulating intermediate layer 13 into two electrode body halves 2′, 2″ that are electrically insulated from each other. In contrast, FIG. 2b shows a monopolar electrode body 2 with just one electrical contact sleeve

The electrode bodies 2 shown in FIGS. 2a, b each have a spherically dome shape along the first axial portion 5 which seamlessly and smoothly transforms into a straight cylindrical outer shape along the second axial portion 6.

The cross-sectional surfaces of the electrode bodies 2 which respectively are spherically formed along the first axial portion 5, correspond to circular areas each with continuously increasing circle diameters up to a circle diameter that corresponds to the diameter of the straight cylindrical outer shape along the second axial portion 6. The associated longitudinal sections through the electrode body 2 thus represent semicircular areas in the first axial portion 5.

FIG. 3a shows a shape variant designed of the electrode body 2, which in contrast to the spherical dome shape in accordance with FIGS. 2a, b, also has a smaller dome shape 10. The mamilla-like dome 10 is for supporting the pushing apart of two anatomical structure that are connected to each other by at least one of a connective tissue, vascularised tissue, or an avascularised tissue layer.

FIG. 3b shows a view from above of the distal end 9 of the electrode body 2 in proximal projection. The circular design of the mamilla-like dome shape can be seen from this illustration. The transition between the mamilla-like dome 10 and the remaining contour of the electrode body 2 within the first axial portion 5 takes place seamlessly and smoothly, that is the surface of the electrode body 2 is continuously differentiable at every point.

The shape of the dome-shaped electrode body 2 can diverge from the spherical dome-shaped design in accordance with the forms of embodiment in FIGS. 1 and 2.

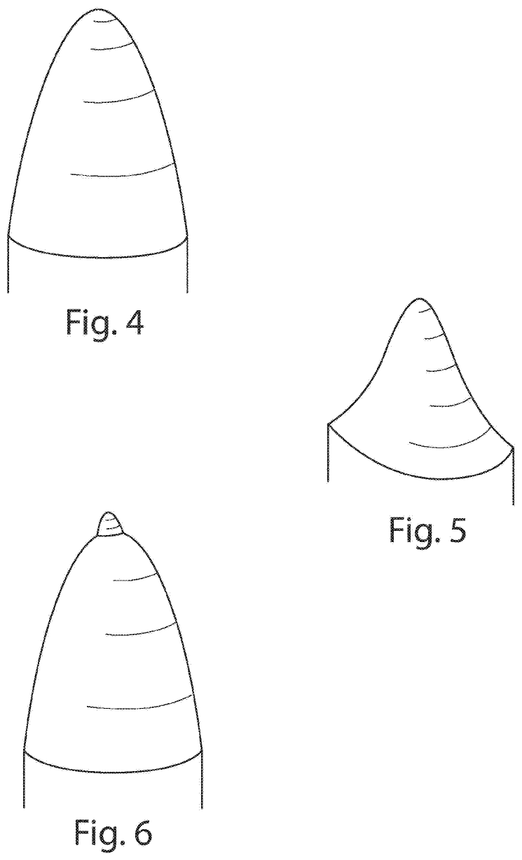

FIGS. 4 to 6 show alternative spatial shapes for designing the electrode body 2 more particularly along the first axial section 5. FIG. 4 shows the spatial shape of a paraboloid and FIG. 5 is a replica of a Gaussian radiation intensity distribution which identically or approximately and in sections corresponds with the spatial shape of an ellipsoid.

FIG. 6 shows the special shape of a paraboloid, comparable with the illustration in FIG. 4, but which is supplemented with a mamilla-like additional dome shape 10 on the distal end of the electrode body 2.

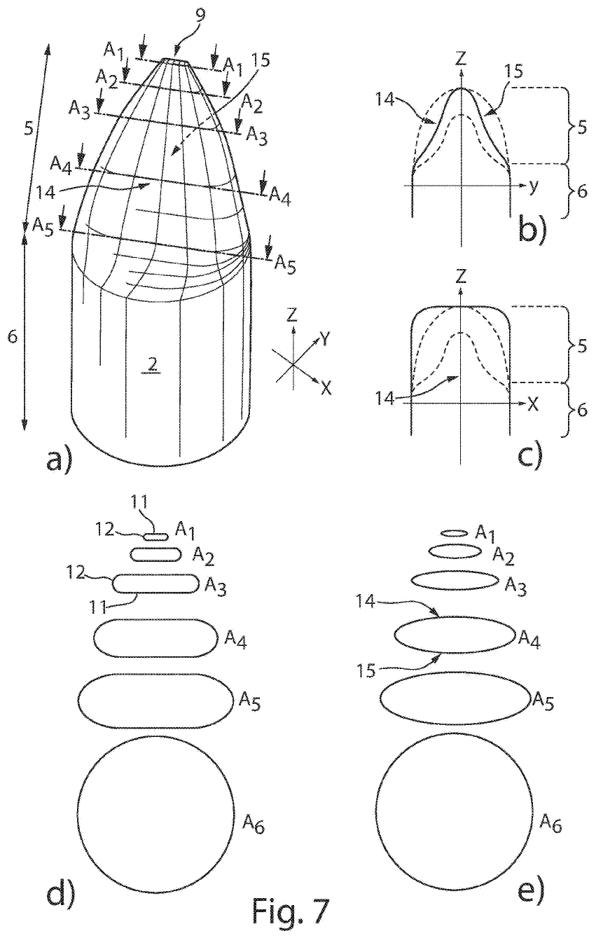

FIG. 7a shows a perspective view of a further design form for shaping the electrode body 2 at least along the first portion 5. In this case the dome-shaped design of the electrode body is shovel-like or roundly flattened. Through the flattening of the electrode body 2 within the first portion 5 along the y-axis, see the x-y-z coordinate system shown in FIG. 7a, two opposite, flattened electrode surfaces 14, 15 are formed along the y-axis which are each moved in parallel, or largely in parallel between two tissue layers to the separated for the purpose of pushing apart both anatomical layer structures.

FIGS. 7b and 7c each show sectional views through the electrode body 2, respectively along the section plane z-y. See FIG. 7b along the section plane z-x. See FIG. 7c, on the continuous line in each case. The profile sections indicated with dashed lines in FIGS. 7b and 7c show, in a non-restrictive manner, variations for designing the spatial shape of the electrode body 2 illustrated in FIG. 7a.

FIGS. 7d and 7e also show possible alternative cross-sectional shapes A1 to A5, in the order of the section planes shown FIG. 7a.

In the case of the cross-sectional shapes in accordance with FIG. 7d, the electrode body 2 has cross-sectional shapes A1 to A5 in the first axial portion 5, which each have a straight section 11 as well as curved sections 12. With increasing distance from the distal end 9, the cross-sectional shapes A1 to A5 morphologically approximate a circular cross-section. The straight sections 11 are each assigned to the flattened electrode surfaces 14 15.

In the case of the cross-sectional shapes A1 to A5 illustrated in FIG. 7e, these are elliptical cross-sections whose cross-section dimensions continuously increase from the distal end 9 towards the proximal end. The slightly curved elliptical sections are assigned to the flattened electrode surfaces 14, 15.

In this case too, the elliptical cross-sectional shaped morphologically transition into a circular cross-section A6, which corresponds to the outer hollow cannula cross-section of the probe 1.

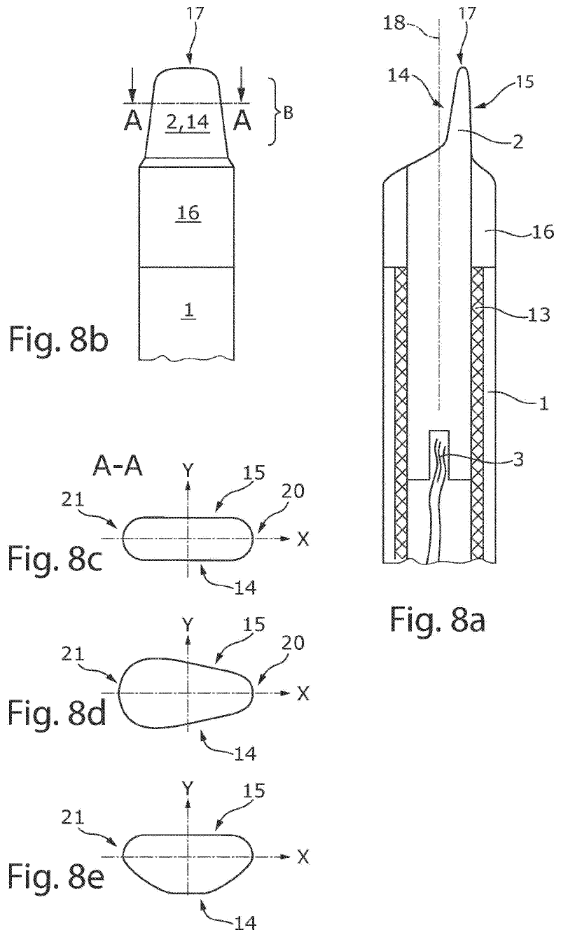

FIG. 8a shows a longitudinal section through a further form of embodiment for the enucleation of intracorporeal tissue regions with a probe 1 designed as a rigid hollow cannula, mounted at the distal end of which is an electrode body 2 to which electrical energy can be supplied via at least one electrical line 3 running in the longitudinal extent of the probe 1. By way of an electrical insulation layer 13 incorporated within the probe 1, the electrode body 2 is galvanically decoupled from the metallic probe wall. In addition, an electrically insulating ceramic sleeve body 16 surrounds the electrode body 2 distally projecting beyond the hollow cannula 1. The ceramic sleeve body 16 is flush with the outer contour of the hollow cannula 1. Area B of the electrical body 2, which distally projects beyond the electrically insulating ceramic sleeve body 16, is flattened, like the shape of a spatula, and comprises two electrode surfaces 14, 15, the shape of which can be seen by jointly looking at the longitudinal view in accordance with FIG. 8a and the side view in accordance with FIG. 8b which is turned about 90° with regard to FIG. 8a. Furthermore, possible spatial embodiments of the electrode body 2 are also evident with reference to the FIGS. 8c to 8e.

The distal end 17 of the spatula-shaped, flattened electrode body 2 has a distally rounded contour, to which two electrode surfaces 14, 15 seamlessly adjoin. The distal end 17 is also arranged eccentrically with regard to the longitudinal axis of the probe 18. The electrode surface 15 ends in a largely contour-maintaining manner on the outer wall of the proximally extending electrode body 2. The electrode surface 14, on the other hand, is curved in a shovel-like manner and radially seamlessly adjoins the face edge of the ceramic sleeve body 16, which is designed to maintain the contours of the shovel shape.

In practical application of the probe, the rounded end 17 ensures sparing displacement and separation of two tissue layers. The shape of the two electrode surfaces 14 and 15 as well as the proximally adjoining face-side contours of the ceramic sleeve body 16 allow for spatial distancing of the separated tissue regions.

In FIG. 8c a preferred cross-section through the electrode body 2 along section A-A in FIG. 8b is shown. Both electrode surfaces 14, 15, which are flattened along the y cross-section axis, are flat symmetrically to the x cross-section axis, and on their opposite surface ends along the y-axis are each connected by a rounded, preferably cylindrical surface shape 20, 21.

An alternative cross-sectional shape is illustrated in FIG. 8d. In this case the electrode surfaces 14, 15, are also designed to be symmetrical to the x cross-section axis, but flat and converging. The surface ends of both electrode surfaces 14, 15 are each connected via differently dimensioned cylindrical surface shapes 20, 21 of which the left surface shape 21 in the cross-sectional view according to FIG. 8d, has a greater curvature radius than the opposite surface shape 20. In the case of a lateral movement of the probe in the direction of the thicker surface shape 21, the broader or thicker, rounded surface shape 21 supports the separation process between two tissue layers, avoiding the consequences of cutting in the tissue.

FIG. 8e shows a further, alternative cross-sectional shape. In this case the electrode surface 15 is flat and the opposite electrode surface 14 is convex in design.

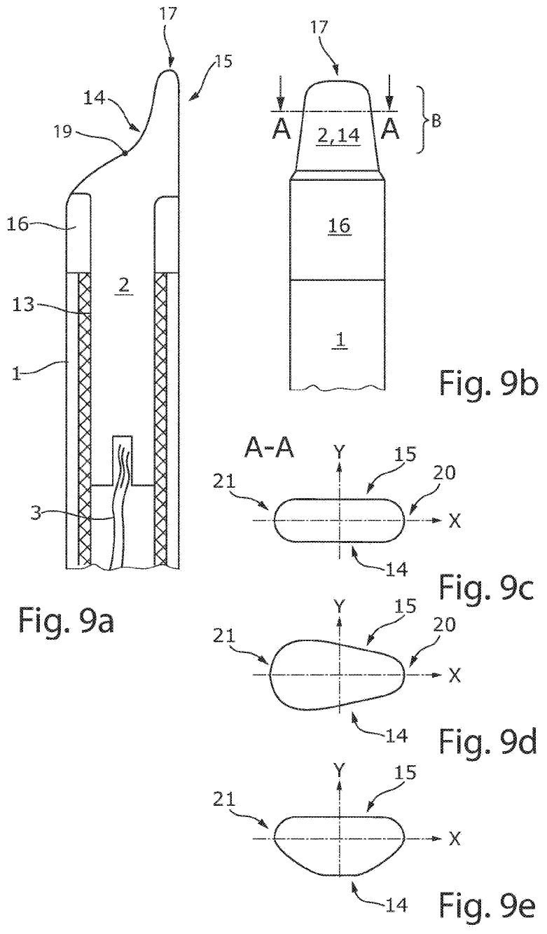

A variant of embodiment modified with regard to the embodiment shown above in FIGS. 8a, b, is illustrated in FIGS. 9a, b, which shows a longitudinal section view and a longitudinal view turned about 90°. In this case the area B of the electrical body 2 distally projecting beyond the ceramic sleeve body 16 radially and axially adjoins the outer wall of the ceramic sleeve body 16 in a flush manner, wherein the electrode surface 15 axially adjoins the outer wall of the ceramic sleeve body 16 in a flush manner, whereas the electrode surface 14 is curved in a shovel shape and has a center of curvature 19. The electrode body 2 shown in FIGS. 9a, b can also assume spatial shapes that are defined by the alternative cross-sections shown in FIGS. 9c to 9e as well as the cross-sections show in FIGS. 8c to 8e.

In all the examples of embodiment illustrated above, the entire surface area of the electrode body 2 is at least one of smoothly polished and honed, at least along area B. Preferably the electrode surface area of electrode body 2, the ceramic sleeve body 16 and the probe 1 are covered with a low-friction coating, preferably with a coating containing PTFE, TPU, polysiloxane or hydrogel.

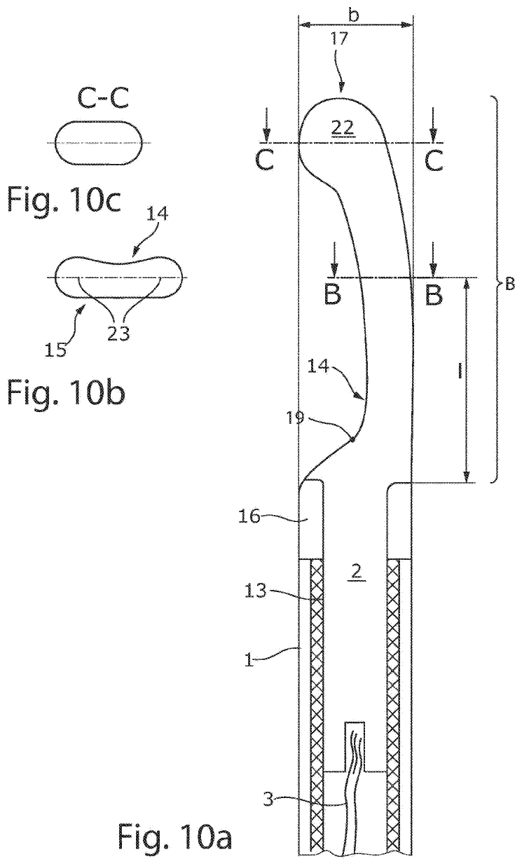

FIG. 10a shows a longitudinal section through a further example of embodiment of a device according to the solution. In order to avoid repetition, components that are at least one of designed and act identically to already mentioned components, and are provided with already used and explained reference numbers.

The electrode body 2, comprising an electrically conductive and dimensionally stable material, preferably a metal or a metal alloy, is enclosed in a mechanically stable, torsion-free and rigid as well as electrically insulated manner within the hollow cannula 1 as well as the adjoining ceramic sleeve body 16. Area B of the electrode body 2, which distally projects from the ceramic sleeve body 16, is spoon-like or shovel-like in design. At its distal end 17, the electrode body 2 has a bulbous and rounded thickening 22. Proximally, the electrode surface 15 flushly adjoins the outer contour of the ceramic sleeve body 16. Via a section 1 the electrode surface 15 extends essentially in parallel, rectilinearly to the longitudinal extent of the ceramic sleeve body 16. Subsequent to this, the electrode surface 15 is convexly curved and at the distal end 17 merges into the bulbous and rounded thickening 22.

The electrode surface 14 is essential concavely formed in a spoon-like manner and on both of its longitudinal sides has bulbous edge contours 23, whose spatial shape can be seen in the cross-sectional view according to 10b along section plane B-B.

Through the bilateral superelevations on the electrode surface 14 resulting from the bulbous edge contours 23 vis-a-vis the concave recess arranged centrally to the longitudinal axis of the probe, this shape provides the electrode body 2 with increased dimensional or bending strength, particularly in the case of forces acting transversely to the longitudinal extent of the probe. The surface contour of the electrode surface 14 resembles the outer contour of a figure eight, whereas the opposite electrode surface 15 is flat.

In the distal area of the bulbous and rounded thickening 22, along the section line C-C visible in FIG. 10a, the electrode body 2 has the oval or elliptical cross-section shape shown in FIG. 10c.

The radial extent or spatial expansion of the electrode body 5, does not project beyond the radial dimension of the hollow cannula 1, which is defined by the outer diameter b, so that it is ensured that the entire probe can be fed unhindered through a working channel of a resectoscope which is dimensionally matched to the hollow cannula.

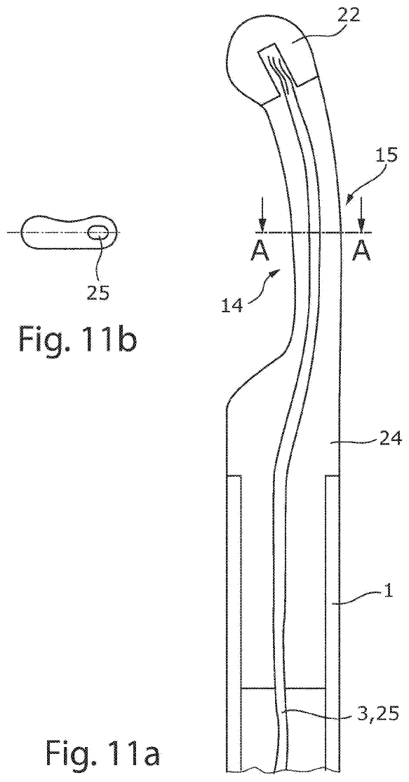

FIG. 11a shows a longitudinal section through a probe, which instead of the ceramic sleeve body 16 and the electrode body 2, as set out above with regard to FIG. 10, has a body 24 that projects into the hollow cannula 1 and is firmly connected to the hollow cannula 1, and that with the exception of the bulbous and rounded thickening 22 made of an electrically conductive material, is made of an electrical insulator, preferably a ceramic or a fiber-reinforced polymer, e.g. GFK, and otherwise has the shape of the electrode body 2 shown and described in the above FIGS. 10a, b. Extending through the body 24, is an electrical line assembly 3 that is connected to the electrically conducting thickening 22.

The cross-sectional view according to FIG. 11b, corresponds to the cross-section through the body 24 along section plane A-A in accordance with FIG. 11a and replicates the cross-section of the electrode body 2 along section plane B-B in accordance with FIG. 10b. A feedthrough channel for 25 for the electrical line assembly 3 in also provided in the cross-section.

The probe illustrated in FIG. 11a allows the limited as-needed application of electrical energy only to the areas of the bulbous and rounded thickening 22, and a thereby achievable coagulation effect on the surrounding tissue. All other surfaces of the body 24 are electrically inactive.

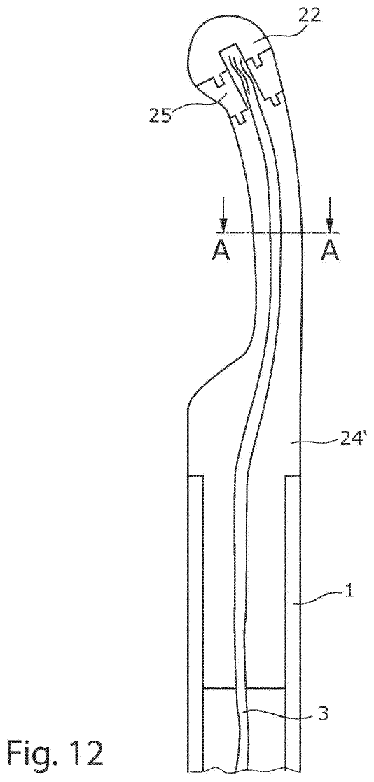

Alternatively to the embodiment of the insulating body 24, FIG. 12 shows a longitudinal section through an example of embodiment, in which the 24′ is made of a metallic, electrical conductive material. The body 24′ is firmly joined to the hollow cannula 1 or connected in one piece thereto. Distally on the metallic body 24′ an electrical insulator 25 is applied, to which, electrically insulated from the metallic body 24′, the bulbous and rounded thickening 22 made of a metallic material is applied, which via the electrical lead assembly 3, can be supplied as-needed with electrical energy. The metallic body 24′, the spatial shape of which essentially corresponds to that of the previously described body 24 in accordance with FIGS. 11a, b or electrode body 2 in accordance with FIG. 10a, possesses a high degree of robustness and bending strength, particularly in the case of one-piece or monolithic embodiment with the hollow cannula 1. Along the section line A-A, the metallic body 24′ has a cross-section equivalent to the cross-section in accordance with FIG. 11b.

### LIST OF REFERENCE NUMBERS

1 Probe, hollow cannula
2 Electrode body
3 Electrical line
4 Guide sleeve
5 First axial portion
6 Second portion
7 Joint
8 Contact sleeve
9 Distal end
10 Mamilla dome shape
11 Straight section
12 Curved section
13 Electrically insulating intermediate layer
14, 15 Electrode body surface
16 Ceramic sleeve body
17 Distal end
18 Longitudinal axis of the probe
19 Center of curvature
20, 21 Surface shape
22 Thickening
23 Bulbous edge contour
24 Electrically insulating bodies
25 Feedthrough channel

1-29. (canceled)

30. A device for the enucleation of intracorporeal prostate tissue regions comprising:
a probe including a rigid hollow cannula and a distal end with at least one freely accessible electrode body to which electrical energy is applied via at least one electrical line running longitudinally along the probe;
the at least one accessible electrode body comprises a dome-shaped electrode surface element with cross-sectional surfaces orientated orthogonally to a longitudinal dimension of the probe with surface areas along first axial portion containing a distal dome end of the at least one accessible electrode body which continuously increase in cross-section as a distance from the distal dome end increases and cross-sectional surfaces including a peripheral edge are continuously differentiable; and
at least in an area of the accessible electrode body distally connected to the probe, the probe body has a bending stiffness, which under an effect of a bending moment of at least 0.1 Nm acting on the electrode body transversely to a longitudinal extension of the probe, does not change shape.

31. The device according to claim 30, wherein:
the peripheral edge of the cross-sectional surfaces of the electrode body is continually curved.

32. The device according to claim 31, wherein:
the dome-shaped electrode surface along the first axial portion has a spatial shape corresponding to a spatial radiation intensity distribution of a laser beam with a Gaussian intensity distribution, a paraboloid or ellipsoid.

33. The device according to claim 30, wherein:
the peripheral edge of the cross-sectional surfaces of the electrode body only has curved and straight peripheral edge sections.

34. The device according to claim 30, wherein:
adjoins the first axial portion of the electrode body is a second axial portion of the electrode body and cross-sectional surfaces orientated orthogonally to the longitudinal extension of the electrode body do not change as distance from the distal dome end increases.

35. The device according to claim 30, wherein:
in the first axial portion the electrode body has longitudinal sections orientated orthogonally to a cross-section surface which is delimited by a continuous peripheral edge.

36. The device according to claim 35, wherein:
the peripheral edge is shaped as one of a circle, a parabola, a partial ellipse or a partial oval.

37. The device according to claim 30, wherein:
the probe transmits at least one of thrust and pressure forces along a longitudinal extension of the probe.

38. The device according to claim 30, wherein:
the hollow cannula is made of a metallic material.

39. The device according to claim 30, wherein:
when in an area of the electrode body distally connected to the probe, the probe has a bending stiffness under an effect of a bending moment of at least 0.3 Nm acting on the electrode body transversely to the longitudinal extension of the probe, dimensions of the probe do not change.

40. The device according to claim 30, wherein:
an area of the electrode body distally connected to the probe extends from its distal electrode tip to a maximum of 30 mm.

41. The device according to claim 30, wherein:
the electrode body is made of one of metal or a metal alloy formed as a monopolar electrode electrically connected with an electrical line extending along the probe or formed as a bipolar electrode with two electrical lines extending along the probe.

42. The device according to claim 30, comprising:
a guide sleeve extending along the probe for feeding a medical instrument in at least one of parallel to the probe and centering and sliding within and along a working channel of a resectoscope.

43. The device according to claim 30, wherein:
at least the electrode surface of the electrode body is polished and honed.

44. The device according to claim 30, wherein:
the electrode body is connected to the probe by a biocompatible, electrically insulating joint.

45. The device according to claim 44, wherein:
at least one of an electrode surface of the electrode body, the joint and the probe is coated with a friction reducing coating.

46. The device according to claim 45, wherein:
the coating comprises PTFE, TPU, polysiloxane or hydrogel.

47. The device according to claim 30, wherein:
along a cross-sectional axis of a cross-section of the electrode body, the electrode body comprises two flattened electrode body surfaces.

48. The device according to claim 47, wherein:
the two flattened electrode body surfaces comprise one of:
both electrode body surfaces have at least one level surface area;
both electrode body surfaces have at least one convexly curved surface area;
one electrode body surface has at least one convexly curved surface area and an other electrode body surface has at least one concavely curved surface area; and
one of the two electrode body surfaces has at least one curved surface area and the other electrode body surface has at least one concavely surface area.

49. The device according to claim 47, wherein:
the electrode body is a spatula shaped.

50. The device according to 47, wherein:
the electrode body has an oval-shaped cross-section which is symmetrical to a longitudinal axis of the oval cross-section.

51. The device according to claim 50, wherein:
the oval-shaped cross-section is not symmetrical to an axis orthogonal to the longitudinal axis.

52. The device according to claim 47, wherein:
the electrode body is shovel shaped with a flattened electrode body surface, which on a proximal side has a straight surface section which adjoins distally a convexly curved surface section; and
the flattened electrode body surface is convexly curved and distally has a bulbous and rounded thickening.

53. The device according to claim 52, wherein:
in axial projection to a rigid hollow cannula, the freely accessible electrode body does not radially protrude beyond the hollow cannula.

54. The device according to claim 52, wherein:
the freely accessible electrode body has a cross-section which at least in sections has an outer shape of a figure eight.

55. The device according to claim 30, wherein:
distally from the rigid hollow cannula, a spatula or shovel-shaped molded body is applied on which distally an accessible electrode body is mounted.

56. The device according to claim 55, wherein:
the body comprises an electrically insulating material on which distally the accessible electrode body is mounted, or
the body is made of an electrically conductive material on which is on an electrical insulator.

57. The device according to claim 55, wherein:
the body is a shovel with a flattened electrode body surface, has a straight section on a proximal side, is convexly curved with an adjoining distally surface section;
an other flattened electrode body surface is convexly curved; and
the freely accessible electrode body has an adjoining bulbous or rounded thickening.

58. The device according to claim 55, wherein:
the body has a cross-section which at least in sections has an outer shape of a figure eight.
